(19) United States
(12) Patent Application Publication
(10) Pub. No.: US 2014/0287512 A1
(43) Pub. Date: Sep. 25, 2014

(54) METHOD OF CULTIVATING CELLS ON MICROCARRIERS IN A BAG

(71) Applicant: GE HEALTHCARE BIO-SCIENCE AB, Uppsala (SE)

(72) Inventors: Christian Kaisermayer, Vienna (AT); Eva Lindskog, Uppsala (SE)

(21) Appl. No.: 14/118,319

(22) PCT Filed: May 15, 2012
(86) PCT No.: PCT/SE2012/050516

§ 371 Date: Nov. 18, 2013

(30) Foreign Application Priority Data
May 16, 2011 (SE) 1150448-7

(51) Int. Cl. C12N 5/071

(52) U.S. Cl. CPC C12N 5/0602
USPC 435/456; 435/364; 435/369; 435/350; 435/289.1

(57) ABSTRACT

A method of cultivating cells in a bag is disclosed, which comprises the steps of:

a) providing a sterile flexible bag comprising a filter material which is attached to a wall of the culture bag, wherein the filter material delimits a chamber inside the bag and wherein the chamber is fluidically connected to a first port in a wall of the bag,

b) providing agitation means for the bag,

c) introducing cell culture medium, microcarriers and cells to the bag,

d) cultivating cells in the bag with agitation provided by the agitation means, allowing formation of a suspension of microcarriers with immobilized cells and supplying at least one gas via a second port (not shown) in a wall of the bag,

e) removing cell debris and/or free cells together with liquid from said suspension of microcarriers through the filter material and the first port.

### TECHNICAL FIELD OF THE INVENTION

The present invention relates to cultivation of cells in bags and in particular to perfusion cultivation of cells on microcarriers. The invention also relates to bags for perfusion cultivation of cells on microcarriers.

### BACKGROUND OF THE INVENTION

The bio-processing industry has traditionally used stainless steel systems and piping in manufacturing processes for fermentation and cell culture. These devices are designed to be steam sterilized and reused. Cleaning and sterilization are however costly labour-intensive operations. Moreover, the installed cost of these traditional systems with the requisite piping and utilities is often prohibitive. Furthermore, these systems are typically designed for a specific process, and cannot be easily reconfigured for new applications. These limitations have led to adoption of a new approach over the last ten years—that of using plastic, single-use disposable bags and tubing, to replace the usual stainless steel tanks.

In particular bioreactors, traditionally made of stainless steel, have been replaced in many applications by disposable bags which are rocked to provide the necessary aeration and mixing necessary for cell culture. These single-use bags are typically provided sterile and eliminate the costly and time-consuming steps of cleaning and sterilization. The bags are designed to maintain a sterile environment during operation thereby minimizing the risk of contamination.

Commonly used bags are of the “pillow style,” mainly because these can be manufactured at low cost by seaming together two flexible sheets of plastic. Three-dimensional bags have also been described, where further sheets may be used to create wall structures.

One of the successful disposable bioreactor systems uses a rocking table on to which a bioreactor bag is placed. The bioreactor bag is partially filled with liquid nutrient media and the desired cells. The table rocks the bag providing constant movement of the cells in the bag and also efficient gas exchange from the turbulent air-liquid surface. The bag, typically, has at least one gas supply tube for the introduction of air, carbon dioxide, nitrogen or oxygen, and at least one exhaust gas tube to allow for the removal of respired gases. Nutrients can be added through other tubes.

Traditionally, cell culture has been operated in a batch mode. In batch operation, the bioreactor is seeded with a small amount of cells and the cells are grown to higher density. The cells produce the product of interest and eventually die due to lack of nutrients or to build-up of toxic metabolites, at which point the culture is harvested. This method has several drawbacks-firstly, a large fraction of nutrients are wasted in simply growing up cells and are not used directly for making the product; secondly, product formation is often inhibited due to the build-up of toxic metabolic by-products; and lastly critical nutrients are often depleted leading to low final cell densities and consequently lower product yields.

It has long been recognized that perfusion culture offers better economics for some processes. In this operation, cells are retained in the bioreactor, and toxic metabolic by-products are continuously removed. Feed, containing nutrients is continually added. This operation is capable of achieving high cell densities and more importantly, the cells can be maintained in a highly productive state for weeks-months. This achieves much higher yields and reduces the size of the bioreactor necessary. It is also a useful technique for cultivating primary or other slow growing cells. Perfusion operations have tremendous potential for growing the large number of cells needed for human cell and genetic therapy applications.

Many cell types do not grow well in free suspension and in larger scale cultivations they are normally grown on microcarriers, i.e. particles providing surfaces for the cells to grow on.

Vero and MDCK cells are widely used for production of viral vaccines such as polio, measles, influenza, rabies or entero virus. For large scale cultivation the cell lines are usually grown on microcarriers in volumes up to 6000 liters. Virus yields often correlate with the cell concentration and therefore methods strive for obtaining high cell counts before infection. However, nutrient supply and metabolic state of the cells also have an important impact on the virus concentration achievable. It has been observed that, due to a so called “cell density effect”, increasing cell concentrations above a certain level does no longer improve the virus yield. This was suspected to be due to inhibitors that accumulated under batch and fed-batch conditions (Yuk et al Cytotechnology 51(3), 183-192, 2006). It has been shown that adequate supply of nutrients and removal of waste products by cell cultivation in perfusion systems can increase the virus yield five- to seven-fold compared to batch cultivation (Rourou et al Vaccine 25(19), 3879-3889, 2007). During microcarrier cultivation, non-viable cells can detach from the microcarriers and cell debris can be formed as a consequence of physical attrition or cell lysis. It is desirable to remove both the debris particles and the free cells as they may release proteases, cytokines, DNA etc. which have a negative influence on the viability of the immobilized cells in the culture. Additionally operating life of the cell retention will be improved if the particles are removed from the bioreactor rather than accumulated during the process.

Perfusion culture of cells in free suspension can be carried out in rocking bags, e.g. using the bag designs described in U.S. Pat. No. 6,544,788 or U.S. 20110020922. Here, a small-pore tortuous filter in the bag retains the cells and allows particle-free liquid to be removed from the culture. These constructions are however not suitable for perfusion culture of cells on microcarriers, where it is desirable to remove particles such as cell debris and free cells in addition to the liquid. Hence, there is a need for new methods and constructions allowing perfusion culture in bags with microcarriers.

### SUMMARY OF THE INVENTION

One aspect of the invention is to provide a method allowing efficient perfusion culture of cells on microcarriers. This is achieved with a method as defined in claim 1. One advantage of this method is that it allows efficient removal of cell debris and free cells during the cultivation.

A further aspect of the invention is to provide a construction allowing efficient perfusion culture of cells on microcarriers. This is achieved with a cell culture bag as defined in claim 13. One advantage of this construction is that it allows efficient removal of cell debris and free cells.

A third aspect of the invention is to provide a bioreactor allowing efficient perfusion culture of cells on microcarriers. This is achieved with a bioreactor as defined in claim 24. One advantage of this construction is that it allows agitation by rocking, which is particularly useful in the removal of cell debris and free cells during the cultivation.

Further suitable embodiments of the invention are described in the depending claims.

### BRIEF DESCRIPTION OF THE DRAWINGS

FIG. 1 shows a cell culture bag according to the invention—a) side view, b) top view.

FIG. 2 shows an enlarged side view of the bag of FIG. 1.

FIG. 3 shows the bag of FIGS. 1 and 2 mounted on a movable support.

FIG. 4 shows a cell culture bag according to the invention—a) side view, b) top view.

FIG. 5 shows enlarged views of the bag of FIG. 4—a) side view, b) end view.

FIG. 6 shows end views of two alternative arrangements of the bag in FIGS. 4 and 5.

FIG. 7 shows a cell culture bag according to the invention—a) side view, b) top view

FIG. 8 shows enlarged views of the bag of FIG. 7—a) side view, b) top view.

### DEFINITIONS

The term “port” as used herein means an opening in an inflatable bag, adapted for transport of material into or out of the bag or for mounting of transducers. Ports are often equipped with tube fittings.

The directional terms “top”, “bottom”, “above” and “below” are herein defined with respect to the operational use of the bag, when it is in a generally horizontal orientation, with cell/microcarrier suspension mainly contacting a bottom wall of the bag and gas mainly contacting a top wall of the bag.

The term “immobilized” herein means that a cell is attached to a microcarrier particle, either by adhesion to a surface of the microcarrier or by entrapment in a pore of the microcarrier.

The term “free cell” herein means a cell which is not immobilized.

### DETAILED DESCRIPTION OF EMBODIMENTS

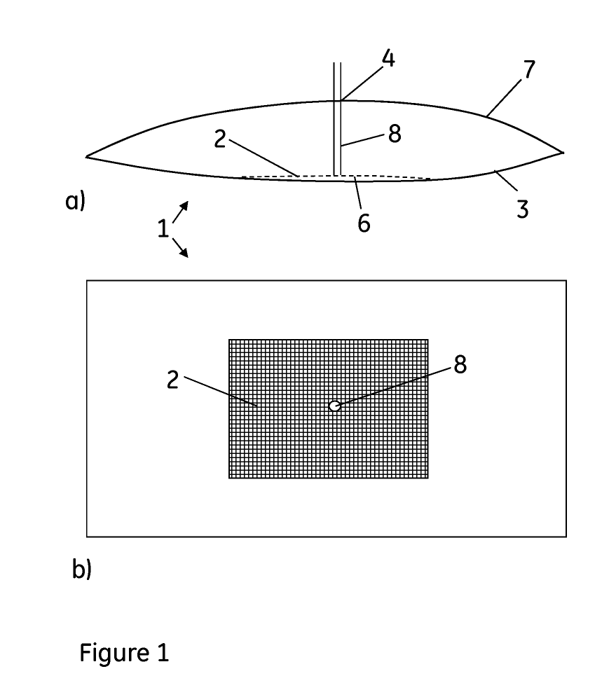

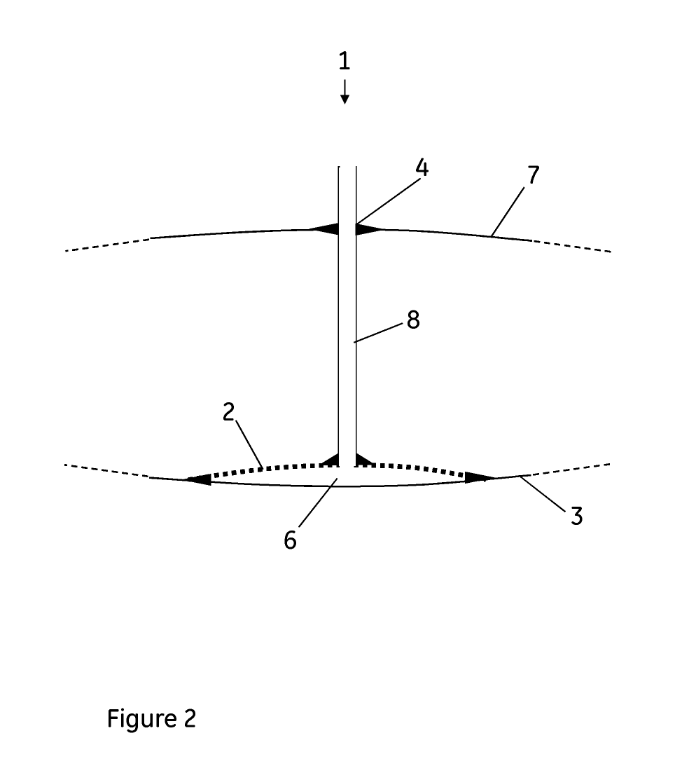

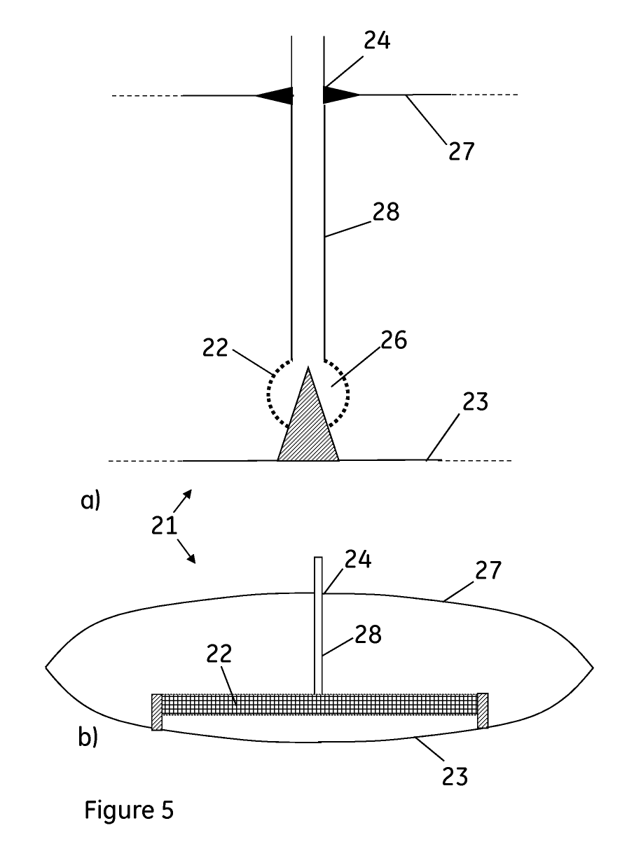

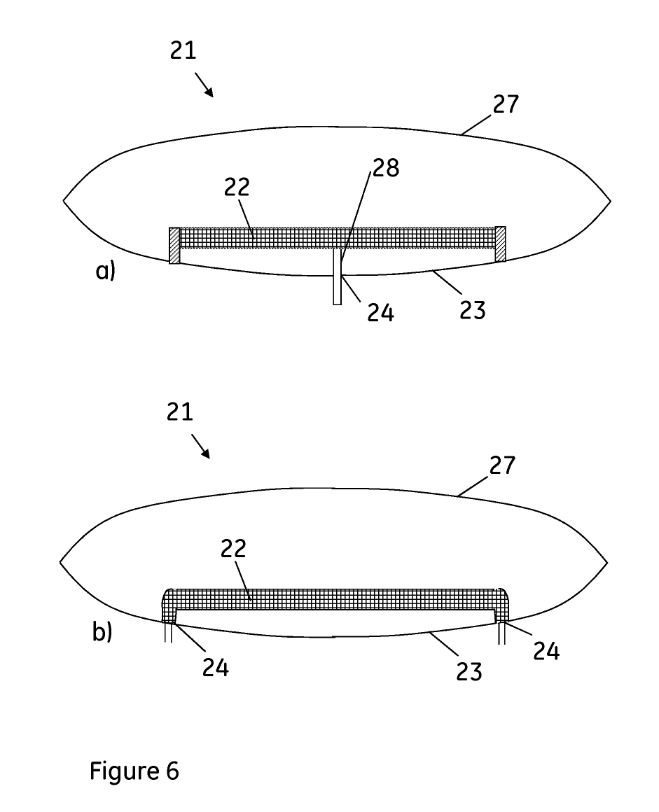

In a first aspect illustrated by FIGS. 1-8, the present invention discloses a method of cultivating cells in a bag, which comprises the steps of:
a) providing a sterile flexible bag 1;21;41 comprising a filter material 2;22;42 which is attached to a wall 3,7;23,27;43,47 of the culture bag, wherein the filter material 2;22;42 delimits a chamber 6;26;46 inside the bag and wherein the chamber 6;26;46 is fluidically connected to a first port 4;24;44 in a wall 3,7;23,27;43,47 of the bag,
b) providing agitation means for the bag,
c) introducing cell culture medium, microcarriers and cells to the bag,
d) cultivating cells in the bag with agitation provided by the agitation means, allowing formation of a suspension of microcarriers with immobilized cells and supplying at least one gas via a second port (not shown) in a wall of the bag,
e) removing cell debris and/or free cells together with liquid from said suspension of microcarriers through the filter material 2;22;42 and the first port 4;24;44.

The sterile flexible bag 1;21;41 can be prepared from two flexible sheets joined along their edges to form a “pillow-style bag” or it can be a three-dimensional bag prepared from more than two sheets and having side walls. The flexible sheets may comprise thermoplastics such as polyethylene, ethylene-vinyl acetate copolymers, polyesters, fluoropolymers etc, either in the form of homogeneous films or as multilayer laminates, which may further comprise barrier layers, adhesive layers etc. The sterilization of the bag can be achieved by exposure to radiation, e.g. gamma or electron beam radiation, or by autoclaving.

The chamber 6;26;46 inside the bag is delimited by the filter material 2;22;42 and is attached to the inside of a wall 3,7;23,27;43,47 of the bag, such as a bottom wall 3;23;43. The chamber is sealed so that liquid and smaller particles such as free cells and cell debris can only pass into the chamber via the pores of the filter material 2;22;42, retaining the larger microcarrier particles in the bag outside the chamber. Due to the attachment of the chamber 6;26;46 to the bag wall 3,7;23,27;43,47, it is immobilised in the bag and cannot cause attrition of the cells through movement against the bag walls. It is also contemplated that the bag wall may form one wall of the chamber 6;26;46. The fluidic connection of the chamber to a first port 4;24;44 in a bag wall enables the removal of non-desired cell debris and free cells from the cell culture together with liquid. This connection may be achieved in several ways, e.g. by tubing 8;28;48 inside the bag from the chamber 6;26;46 to a port in a suitable bag wall 3,7;23,27;43,47 or by a port in the bag wall at the location where the chamber is attached to the bag wall or where the bag wall serves as a wall of the chamber.

The cell culture medium, the microcarriers and the cells can be introduced to the bag via tubing and suitable ports (not shown) in the bag according to methods known in the art. The cells can either be supplied as a cell inoculate or as immobilized cells on the microcarriers. The cultivation can proceed according to well known methods, with standard adjustments of agitation intensity, temperature, gas flow rates etc. to provide suitable cultivation conditions for the particular cell line-microcarrier combination. The bag can be inflated during cultivation, which allows for more efficient removal of cell debris and free cells due to improved agitation.

The removal of liquid and cell debris/free cells can be performed continuously, intermittently or during a specific part of the cultivation, e.g. before infection with a virus or as part of a harvest step at the end of the cultivation.

An advantage of having the chamber attached to a bag wall is that when the fluid in the bag is agitated, the suspension of microcarriers and cells will flow over the filter material so that particles smaller than the pore openings of the filter material can pass through the openings and be removed. If the intensity of agitation is sufficient to prevent formation of a filter cake on the filter material, the passage of cell debris and free cells will be much facilitated. The determination of a suitable intensity of agitation can be made in a simple cultivation experiment where liquid is withdrawn from the chamber and the presence of significant amounts of particulates in the liquid is assessed by visual/microscopic observation or by measuring the turbidity of the liquid according to standard methods. The intensity of agitation can then be adjusted to a level where a steady amount of particulates in the liquid is observed, which is an indication that no filter cake is formed. A further advantage of adjusting the intensity of agitation to prevent the formation of a filter cake is that the growth of cells on the filter surface is minimized, which prevents or at least delays the blockage of the filter pores by growing cells.

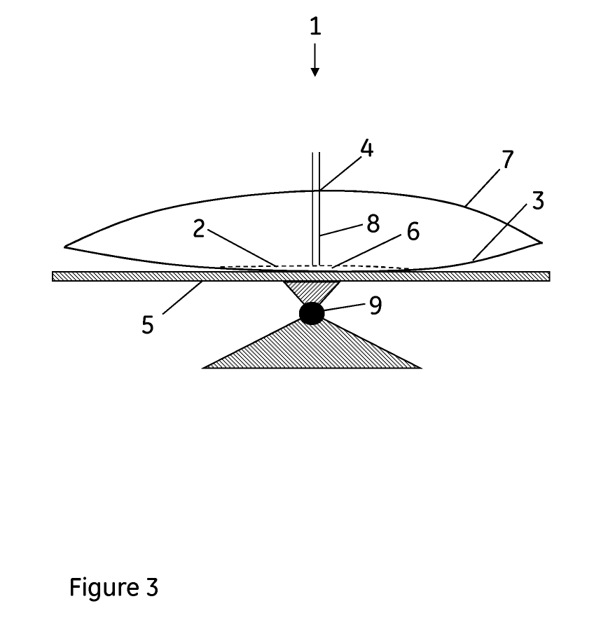

In some embodiments, illustrated by FIG. 3, the agitation means comprises a movable support 5 and step b) comprises mounting the bag 1;21;41 on this movable support. The movable support can e.g. be a plate, trough or container on or in which the bag is placed and, if needed, secured. The movable support can be pivotably mounted on a generally horizontal axis 9, in which case a rocking motion around the axis can be performed. It can also be mounted on a generally vertical excenter axis to provide agitation by orbital motion or on a tilting gyrating axis to provide agitation by gyration. The movable support can also be oscillated in e.g. a generally horizontal direction to provide agitation by oscillation. It is also possible to arrange the movable support to provide combinations of more than one mode of motion. The shape of the bag can be adapted to suit a particular mode of motion. As non-limiting examples, a generally rectangular bag may be used e.g. in conjunction with rocking or horizontally oscillating motion and a bag with generally circular cross section may be used e.g. with gyration or orbital motion.

In certain embodiments the filter material 2;22;42 has a pore size rating between about 30 microns and about 200 microns, such as 70-200 or 90-150 microns. Typical microcarrier particles have diameters in the range of 100-400 microns, while free cells can be around 10-20 microns and cell debris particles range from a few microns and downwards. To achieve essentially complete retention of the microcarrier particles and a high clearance of free cells and cell debris it is advantageous if the pore size rating is smaller than the low end of the particle size distribution of the microcarriers used and significantly higher than the size of the free cells, e.g. at least two to ten times higher. The pore size rating of a filter refers to the ability of the filter to retain particles of different sizes and is typically tested by challenging the filter with narrow size distribution glass beads of different diameters. Pore size ratings are usually supplied by the manufacturers of filter materials, but if no data are available, the following method can be used: A circular filter material disk (e.g. 90 mm diameter) is clamped in a filter holder (e.g. Whatman/GE Healthcare art. No. 1960-009) and a 0.1 wt % suspension of glass beads in 0.1% aqueous Triton X-100 (wetting and dispersing agent, available from e.g. Sigma Aldrich) is prepared. An overhead propeller is arranged in the filter holder about 5 mm above the filter surface and, with 200 rpm agitation, 400 ml of the glass bead suspension is poured into the filter holder and allowed to pass the filter by gravity. The first 200 ml of the filtrate are collected and the concentration of glass beads is determined, e.g. by filtering the suspension on a 0.45 micron membrane filter and weighing the filter. The smallest bead size which is retained to at least 90% is designated as the pore size rating of the filter. Suitable glass beads can be obtained as “sieve standards” from e.g. Whitehouse Scientific Ltd (UK).

In some embodiments the filter material 2;22;42 comprises a mesh filter. Mesh filters are woven or knitted materials with regular openings between the threads. Woven sieving cloths constitute a commonly available type of suitable mesh filter, where monofilament threads are woven with high precision to form thin webs with monodisperse mesh openings and high open areas. The variability in mesh opening size can typically be less than about 5% and the open area can be at least about 20%, such as 25-70%, to minimize the risk of blockage. The mesh filter may comprise polymer threads, such as e.g. polyethylene, polypropylene, polyester or polyamide threads or it may comprise metal threads, such as e.g. stainless steel or bronze threads. The weave pattern of the mesh filter may be square weave, in which case the mesh openings are quadratic and the pore size rating of the filter is equal to the side length of the quadratic openings. It is also possible to use a rectangular weave pattern, where the mesh openings are rectangular and the pore size rating is equal to the width of the rectangular openings. Further possibilities are to use twilled or Dutch weave mesh filters which have elongated openings of more complex shape. The elongated mesh openings in e.g. rectangular, twilled or Dutch weave mesh filters may typically have a length to width ratio of at least about 2. Suitable mesh filters are available from e.g. Membrane Solutions LLC (USA), Wongi Mesh AG (Switzerland), Share Filtration (China), Normesh Ltd (UK), Screen Technology Group Inc (USA) or Spörl KG (Germany).

An advantage of using a mesh filter is that the mesh openings straight through the material facilitate the passage of free cells and cell debris through the material, particularly in comparison with tortuous pore materials such as sintered frits, nonwoven filters and microfilter membranes. Hence it is easier to avoid pore blockage. An advantage of using mesh filters with elongated openings is that they also reduce the risk of pore blockage by spherical microcarrier particles and hence may allow higher flow rates per unit filter area.

In certain embodiments the filter material can be cell-repelling, i.e. with surface properties such that cells do not attach to and grow on the filter. This has the advantage that the filter is not plugged by growth of cells on the filter material during cultivation. One way to achieve a cell-repelling filter is to use a hydrophobic filter material, as cells do not thrive on hydrophobic surfaces. Hydrophobic surfaces can in this context be defined as having a water contact angle higher than about 60 degrees, such as at least 70 degrees or at least 80 degrees. The contact angle is measured by the goniometer sessile drop method on a flat surface of the material in question. Polymer filters may be heat-pressed in order to create a suitable surface for contact angle measurement. Typical water contact angle values for non-modified polymer materials are given in Table 1:

TABLE 1

| Polymer | Water contact angle (degrees) |
|---|---|
| Nylon 6 | 63 |
| Nylon 66 | 68 |
| Polyethylene terephthalate (PET) | 72 |
| Polyethylene | 96 |
| Polypropylene | 102 |

Hydrophilizing treatments like corona or air/oxygen plasma treatment facilitate growth of cells on polymer materials and should be avoided for the filter material.

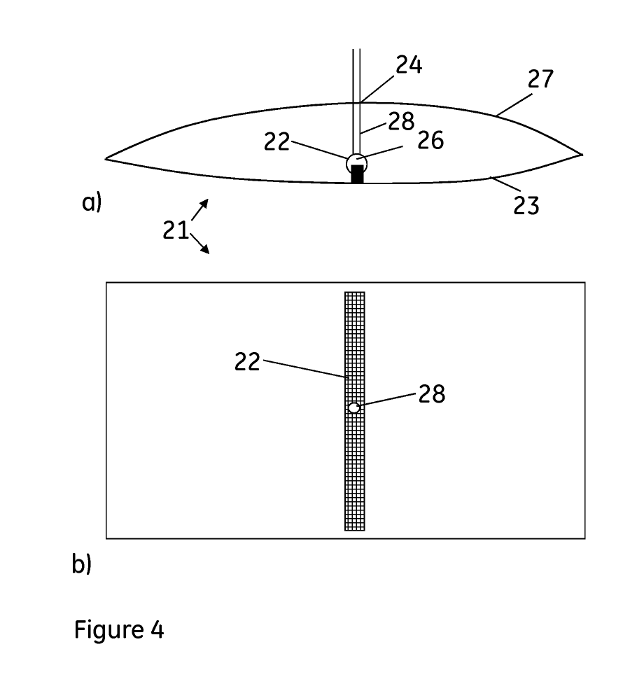

The filter material may in some embodiments be corrugated, in order to increase the filter area and to improve the flow over the filter surface and thus further reduce filter cake formation. The corrugation pattern may comprise essentially parallel ridges and valleys, which may extend in a direction generally parallel to the flow direction, e.g. in a direction perpendicular to the axis 9 when agitation is provided by rocking around this axis. The ridges and valleys may also, particularly in the embodiments illustrated by FIGS. 1-3, extend in a direction generally parallel with a plane defined by the bottom wall 3;23;43 of the bag. In the tubular embodiments illustrated by FIGS. 4-8, the ridges and valleys may extend around the tube(s) in a direction generally perpendicular to the length axis of the tube(s).

In some embodiments the cells comprise animal cells. These can be e.g. mammalian cells, such as cells that are useful for production of recombinant proteins e.g. CHO or HEK cells or cells suitable for the production of virus particles, either as antigens for vaccines or as gene therapy vectors, e.g. Vero or MDCK cells, or they can be cells that in themselves are useful for therapeutic or analytical purposes, such as stem cells or progenitor cells. In cultivation for production of virus particles, a step of infecting the cells with a virus may be included. When the purpose of cultivation is to produce cells as such, e.g. stem cells or progenitor cells, the method may include between steps d) and e) a step of releasing the cells from the microcarriers. The cells can be released e.g. by treatment with proteases like trypsin or collagenase, alternatively cells can also be released by treatment with chemicals, especially chelating agents like EDTA. After release the cells can be recovered via the filter material, while the microcarriers are retained in the bag.

In some embodiments the microcarriers have a density of 1.02-1.05 g/ml and an average particle size of 100-400 micron. Such microcarriers are easily suspended and are suitable for use in flexible bag bioreactors, where the agitation is provided e.g. by rocking motion. The microcarriers may comprise hydrogel materials such as polysaccharides or gelatine, e.g.

cellulose or crosslinked dextran hydrogel beads of average particle size 125-280 micron. The materials are typically treated to make the surfaces suitable for cell adhesion and cell growth, e.g. by introduction of positively charged groups or by coating with cell adhesion-mediating proteins such as collagen. Specific examples of suitable microcarriers are the different grades of Cytodex crossslinked dextran beads and Cytopore cellulose beads, both available from GE Healthcare.

In a second aspect, illustrated by FIGS. 1-8, the invention discloses a cell culture bag 1;21;41, manufactured from a flexible material, comprising microcarriers and cells and which further comprises a filter material 2;22;42 attached to a wall 3,7;23,27;43,47 of the culture bag. The filter material delimits a chamber 6;26;46 inside the bag, which is fluidically connected to a port 4;24;44 in one of the walls 3,7;23,27;43,47 of the bag. This bag can be used for the cell cultivation methods of the first aspect as disclosed above and an advantage is that during use, the cell and microcarrier suspension flows over the filter material, allowing the passage of particles smaller than the filter pore size rating to pass through the filter and to be removed via the port.

As discussed above, the bag can be “pillow-style” or three-dimensional and prepared from two or more sheets of thermoplastic films, e.g. multilayer laminates. It can be supplied pre-sterilised, e.g. by radiation sterilisation or autoclaving.

The chamber 6;26;46 inside the bag is delimited by the filter material 2;22;42 and can be attached e.g. to the inside of a bottom wall 3;23;43 of the bag. The chamber can be sealed e.g. by heat-sealing or adhesive sealing and liquid and smaller particles such as free cells and cell debris can then only pass into the chamber via the pores of the filter material 2;22;42, while the larger microcarrier particles are retained in the bag outside the chamber. The chamber 6;26;46 is immobilised relative to the bag by the attachment to the bag wall 3,7;23,27;43,47, and thus cannot cause attrition of the cells through movement against the bag walls. It is also contemplated that the bag wall may form one wall of the chamber 6;26;46. The fluidic connection of the chamber to a port 4;24;44 in a bag wall may be achieved in several ways, e.g. by tubing 8;28;48 inside the bag from the chamber 6;26;46 to a suitable bag wall 3,7;23,27;43,47, by a port in the bag wall also serving as attachment point for the chamber or by a port in a portion of the bag wall that also constitutes a wall of the chamber. The port 4;24;44 may be located in a bottom wall 3;23;43 of the bag, in which case the movable support 5 may be designed to accommodate the port and tubing extending from the port outside the bag. The bag may also comprise further ports in the bag walls, e.g. for the flow of gases into and out of the reactor, for introduction of cell culture medium, cells and microcarriers, for sample removal and for various sensors and transducers used during cultivation.

As discussed above, the filter material 2;22;42 can have a pore size rating between about 30 microns and about 200 microns, such as 70-200 or 90-150 microns, it can be cell-repelling, such as hydrophobic, and it can be a mesh filter, optionally with elongated openings, such as with a length to width ratio of at least about 2. The total area of the filter material can be from about 5 to about 25 cm² per L working volume of the bag. The area limitation serves the purpose of minimising the number of contacts between the cell-covered microcarriers and the filter, which have the potential to damage the delicate cells.

The filter material may in some embodiments be corrugated. The corrugation pattern may comprise essentially parallel ridges and valleys, which may extend in a direction generally parallel to the flow direction, e.g. in a direction perpendicular to the axis 9 when agitation is provided by rocking around this axis. The ridges and valleys may also, particularly in the embodiments illustrated by FIGS. 1-3, extend in a direction generally parallel with a plane defined by the bottom wall 3;23;43 of the bag. In the tubular embodiments illustrated by FIGS. 4-8, the ridges and valleys may extend around the tube(s) in a direction generally perpendicular to the length axis of the tube(s).

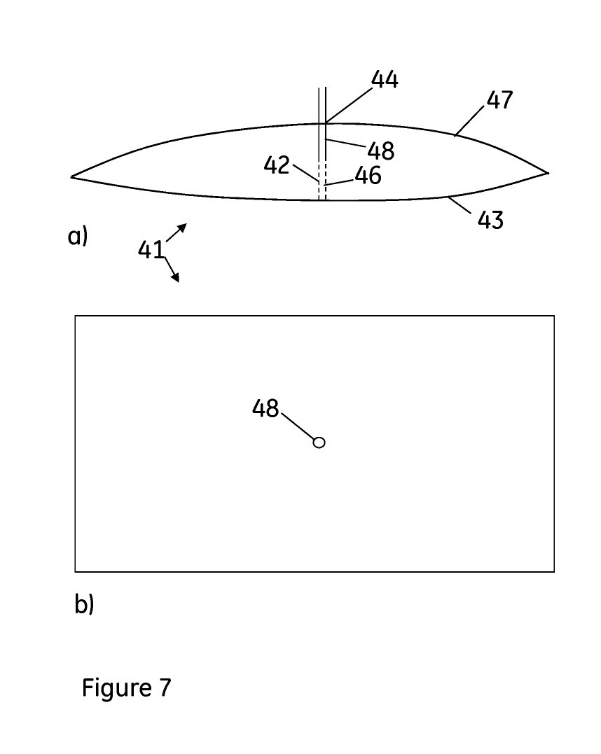

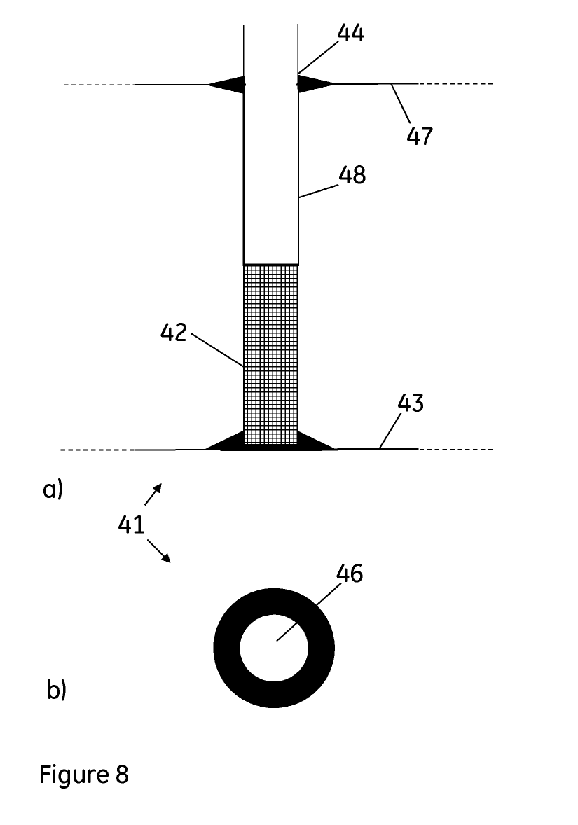

In some embodiments, illustrated by FIGS. 1-3 and 7-8, the chamber 6;46 is delimited by the filter material 2;42 and a bottom wall 3;43 of the bag. This means that a portion of the bottom wall 3;43 also serves as a wall of the chamber 6;46. Such a construction is robust and easily manufactured. The filter material may form either a generally flat sheet as in FIGS. 1-3, a corrugated sheet or a generally tubular structure, e.g. as in FIGS. 7-8.

In certain embodiments, illustrated by FIGS. 4-8, the chamber 26;46 is generally tubular and attached to a bottom wall 23;43 of the bag. The tubular chamber may have e.g. a circular, elliptical or pointed elliptical cross section and it may have at least two attachment points to the bag walls 23,27;43,47, such as to the bottom wall 23;43. With at least two attachment points, the chamber is well fixed to the bag structure and can not move around, causing attrition of the cells against the walls. The tubular structure is easily manufactured, either as a self-supporting filter material structure or from a support structure covered by filter material, and is a convenient way of introducing a suitably large filter area without unnecessary blocking of bag volume. The tubular chamber may further function as a baffle, improving the agitation pattern in the bag.

In some embodiments, illustrated by FIGS. 4-6, the chamber 26 can be generally parallel to the bottom wall 23 and it can e.g. be located at a distance of about 5 to 50 mm above the bottom wall 23. The location close to the bottom wall means that the chamber will be submerged in the suspension even during agitation and hence no suction of air/gas from the chamber will occur. By keeping a separation distance from the bottom wall, the flow is improved, avoiding any filter cake formation in crevices below the chamber. When the bag is mounted on a movable support 5, pivotable around an axis 9, as in FIG. 3, the tubular chamber can be arranged essentially parallel with the axis 9, e.g. on a central part of the bottom wall 23, i.e. where the distance from the projection of the axis 9 on the bottom wall 23 is shorter than the distance to the end of the bag.

In certain embodiments the bag 1;21;41 does not comprise any moving parts. Moving parts such as impellers, floating or freely movable filter compartments etc increase the risk of cell attrition and the constructions according to the invention allow the use of bags without any parts moving in relation to the bag walls.

In a third aspect, the invention discloses a bioreactor comprising a cell culture bag as described above, mounted on a movable support 5, which is pivotable around an axis 9, to allow for agitation by rocking back and forth.

In some embodiments the chamber 6; 26 extends in a direction generally parallel to the axis 9. This provides a good flow over the filter surface and reduces the risk of filter cake formation.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. It is noted that features from different embodiments and aspects may be combined to form further embodiments.

1. A method of cultivating cells in a bag, comprising the steps of:
a) providing a sterile flexible bag (1;21;41) comprising a filter material (2;22;42) attached to a wall (3,7;23,27;43,47) of said culture bag, wherein said filter material delimits a chamber (6;26;46) inside the bag, fluidically connected to a first port (4;24;44) in a wall (3,7;23,27;43,47) of the bag;
b) providing agitation means for said bag;
c) introducing cell culture medium, microcarriers and cells to said bag;
d) cultivating cells in the bag with agitation provided by said agitation means, allowing formation of a suspension of microcarriers with immobilized cells and supplying at least one gas via a second port in a wall of the bag; and
e) removing cell debris and/or free cells together with liquid from said suspension of microcarriers through said filter material (2;22;42) and said first port (4;24;44).

2. The method of claim 1, wherein said agitation means comprises a movable support (5;25;45) and step b) comprises mounting said bag on said movable support.

3. The method of claim 2, wherein said movable support provides agitation by rocking, gyration, oscillation and/or orbital movement.

4. The method of claim 1, wherein the intensity of agitation is adjusted to prevent formation of a filter cake on said filter material (2;22;42).

5. The method of claim 1, wherein said filter material (2;22;42) has a pore size rating between about 30 microns and about 200 microns, such as 90-150 microns.

6. The method of claim 1, wherein said filter material (2;22;42) comprises a mesh filter.

7. The method of claim 1, wherein said filter material (2;22;42) is cell-repelling, such as hydrophobic.

8. The method of claim 1, wherein said cells comprise animal cells, such as mammalian cells, e.g. Vero, HEK, MDCK cells, stem cells or progenitor cells.

9. The method of claim 1, further comprising a step of infecting said cells with a virus.

10. The method of claim 1, further comprising, between steps d) and e), a step of releasing said cells from said microcarriers.

11. The method of claim 1, wherein said microcarriers have a density of 1.02-1.05 g/ml and an average particle size of 100-400 micron.

12. The method of claim 1, wherein said microcarriers comprise cellulose or crosslinked dextran hydrogel beads of average particle size 125-280 micron.

13. A cell culture bag (1;21;41) of flexible material, comprising microcarriers and cells and further comprising a filter material (2;22;42) attached to a wall (3,7;23,27;43,47) of said culture bag, wherein said filter material delimits a chamber (6;26;46) inside the bag, fluidically connected to a port (4;24;44) in a wall (3,7;23,27;43,47) of the bag.

14. The cell culture bag (1;21;41) of claim 13, wherein said filter material (2;22;42) has a pore size rating between about 30 microns and about 200 microns, such as 90-150 microns.

15. The cell culture bag (1;21;41) of claim 13, wherein said filter material (2;22;42) is cell-repelling, such as hydrophobic.

16. The cell culture bag (1;21;41) of claim 13, wherein said filter material (2;22;42) comprises a mesh filter.

17. The cell culture bag (1;21;41) of claim 16, wherein said mesh filter comprises elongated openings, such as with a length to width ratio of at least about 2.

18. The cell culture bag of claim 13, wherein said filter material is corrugated.

19. The cell culture bag (1;41) of claim 13, wherein said chamber (6;46) is delimited by said filter material and a bottom wall (3;43) of the bag.

20. The cell culture bag (1;21;41) of claim 13, wherein said chamber (6;26;46) is fluidically connected by tubing (8;28;48) to a port in a wall (3,7;23,27;43,47), such as a bottom wall (3;23;43) of the bag.

21. The cell culture bag (21;41) of claim 13, wherein said chamber (26;46) is generally tubular and attached to a bottom wall (23;43) of the bag.

22. The cell culture bag (21) of claim 21, wherein said chamber (26) is generally parallel to said bottom wall (23) and optionally located about 5 to 50 mm above said bottom wall (23).

23. The cell culture bag (1;21;41) of claim 13, without moving parts, such as impellers or floating filters.

24. A bioreactor, comprising the cell culture bag of claim 13, mounted on a movable support (5), pivotable around an axis (9).

25. The bioreactor of claim 24, wherein said chamber (6; 26) extends in a direction generally parallel to said axis (9).
